(19) United States
(12) Patent Application Publication
(10) Pub. No.: US 2023/0310460 A1
(43) Pub. Date: Oct. 5, 2023

(54) Low Dose Topical Composition Comprising Triamcinolone Acetonide or Salt Thereof

(71) Applicant: Zenvision Pharma LLP, Mumbai (IN)

(72) Inventors: Sivakumar Venkata Bobba, Navi Mumbai (IN); Amit Manmode, Navi Mumbai (IN); Dhananjay Shinde, Navi Mumbai (IN); Sunil Pophale, Navi Mumbai (IN); Girish Kumar Jain, Navi Mumbai (IN); Shankar Pol, Navi Mumbai (IN)

(21) Appl. No.: 18/023,399

(22) PCT Filed: Aug. 28, 2021
(86) PCT No.: PCT/IB2021/057889

§ 371 Date: Feb. 27, 2023

(30) Foreign Application Priority Data
Aug. 28, 2020 (IN) 202021008558
Aug. 26, 2021 (IN) 202121038728

(51) Int. Cl. A61K 31/58; A61K 9/00; A61K 9/06

(52) U.S. Cl. CPC A61K 31/58; A61K 9/0014; A61K 9/06

(57) ABSTRACT

The present invention relates to a low dose topical composition comprising triamcinolone acetonide or salt thereof in an amount of less than 250 mcg/gm, its process of preparation and its use for the treating/preventing of various types of skin diseases, preferably atopic dermatitis in infants, children, adults and elderly patients.

### FIELD OF THE INVENTION

The present invention relates to a low dose topical composition comprising triamcinolone acetonide or salt thereof, its process of preparation and its use for the treatment of various types of skin diseases.

More particularly the present invention relates to a low dose topical composition comprising triamcinolone acetonide or salt thereof in an amount of less than 250 mcg/gm, its process of preparation and its use for the treating/preventing of various types of skin diseases, preferably atopic dermatitis.

### BACKGROUND OF THE INVENTION

Skin is the layer of usually soft, flexible outer tissue covering the body of a vertebrate animal, with three main functions: protection, regulation, and sensation. Skin diseases are the medical condition that affects the skin, hair, nails and related muscle and glands.

Skin disorders vary greatly in symptoms and severity. They can be temporary or permanent, and may be painless or painful. Some have situational causes, while others may be genetic. Some skin conditions are minor, and others can be life-threatening.

There are many different types of skin disorders which include rashes, dermatoses or skin eruptions. Such rashes, dermatoses or skin eruptions include acute, inflammatory reactions of the skin caused by an allergic or irritant reaction, other forms of eczema, lichen simplex chronicus. Chronic nature includes seborrheic dermatitis, psoriasis, and atopic dermatitis or caused by infection, irritation or aggravation of another condition such as occurs with acne, other rashes, dermatoses or skin eruptions, inflammatory and pruritic manifestations of corticosteroid-responsive dermatoses, contact dermatitis, impetigo, urticarial and scabies.

Typical symptoms of the skin disorders include but not limited to raised bumps that are red or white, a rash, which might be painful or itchy, scaly or rough skin peeling skin, ulcers, open sores or lesions, dry, cracked skin, discolored patches of skin, fleshy bumps, warts, or other skin growths, changes in mole color or size a loss of skin pigment, excessive flushing or the like.

Atopic dermatitis (AD), also known as eczema or atopic eczema, is a type of inflammation of the skin (dermatitis). Atopic dermatitis (AD) is common worldwide. People of all ages from newborns to adults and older live with this condition. Symptoms range from excessively dry, itchy skin to painful, itchy rashes that cause sleepless nights and interfere with everyday life.

Topical corticosteroids have been the mainstay of treatment for atopic dermatitis over the past years, further the cure for atopic dermatitis involves Lifestyle modification, balanced diet intake, self-care measures, phototherapy, wet wrap therapy, use of medications like tacrolimus, pimecrolimus, crisaborole, dupilumab, ciclosporin, methotrexate, interferon gamma-1b, mycophenolate mofetil, and azathioprine or the like.

Triamcinolone Acetonide is a synthetic corticosteroid. Chemically it is [Pregna-1,4-diene-3,20-dione,9-fluoro-11,21-dihydroxy-16,17-[(1 methylethylidene) bis-(oxy)]-, (11β, 16α)-] with the empirical formula C₂₄H₃₁FO₆ and molecular weight 434.50. Triamcinolone Acetonide is represented by compound of structural formula I

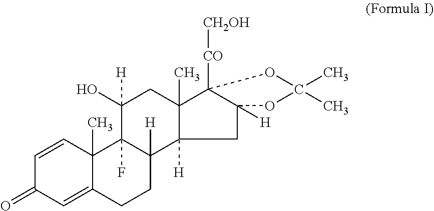

Triamcinolone Acetonide topical cream and ointment with strengths 0.025%, 0.1% and 0.5% (containing 0.25 mg/gm, 1 mg/gm & 5 mg/gm Triamcinolone Acetonide respectively) were approved in USA prior to Jan. 1, 1982 under the trade name “Triamcinolone Acetonide” and were indicated for the relief of the inflammatory and pruritic manifestations of corticosteroid-responsive dermatoses.

The commercially available products or product known in the prior art produces side effects such as burning, itching, irritation, or dryness of skin at site of application, folliculitis, hypertrichosis, acneiform eruptions, hypopigmentation, perioral dermatitis, allergic contact dermatitis, maceration of the skin, secondary infection, skin atrophy, striae and miliaria.

Pediatric patients may demonstrate greater susceptibility to topical triamcinolone-induced HPA axis suppression and Cushing's syndrome than mature patients because of a larger skin surface area to body weight ratio. Hypothalamic-pituitary-adrenal (HPA) axis suppression, Cushing's syndrome and intracranial hypertension have been reported in children receiving topical triamcinolone. Manifestations of adrenal suppression in children include linear growth retardation, delayed weight gain, low plasma cortisol levels, and absence of response to ACTH stimulation. Manifestations of intracranial hypertension include bulging fontanelles, headaches, and bilateral papilledema. Chronic corticosteroid therapy may interfere with the growth and development of children.

Making low dose compositions can present technical and economic challenges that are not present for higher dose formulations.

Hence, there is still need to design improved topical pharmaceutical composition of triamcinolone acetonide to overcome disadvantages/side effects associated with the commercially available high dose topical products available in the prior art.

### Objects of the Invention

It is an object of the present invention to provide a low dose topical composition comprising:
a) Triamcinolone acetonide or salt thereof; and
b) One or more suitable pharmaceutically acceptable excipient.

It is another object of the present invention to provide a low dose topical composition comprising:
a) Triamcinolone acetonide or salt thereof;
b) One or more suitable pharmaceutically acceptable excipient; and
   Wherein said low dose topical composition comprises triamcinolone acetonide or salt thereof in an amount of less than 250 mcg/gm.

It is another object of the present invention to provide a process of preparation of a low dose topical composition comprising triamcinolone acetonide or salt thereof.

It is another object of the present invention to provide a method of using a low dose topical composition comprising triamcinolone acetonide or salt thereof for the treatment of various types of skin disease, preferably atopic dermatitis in infants, children, adults and elderly patients.

### SUMMARY OF THE INVENTION

In one aspect, there is provided a low dose topical composition comprising:
c) Triamcinolone acetonide or salt thereof; and
d) One or more suitable pharmaceutically acceptable excipient.

In another aspect, there is provided a low dose topical composition comprising:
c) Triamcinolone acetonide or salt thereof;
d) One or more suitable pharmaceutically acceptable excipient; and
   Wherein said low dose topical composition comprises triamcinolone acetonide or salt thereof in an amount of less than 250 mcg/gm.

In another aspect, an invention also relates to process of preparation of a low dose topical composition comprising triamcinolone acetonide or salt thereof.

In another aspect, an invention particularly relates to a low dose topical composition comprising triamcinolone acetonide or salt thereof in the form of a stable composition, its process of preparation and its use for the treatment of patients with various skin diseases.

In another aspect, there is provided a method of using a low dose topical composition comprising triamcinolone acetonide or salt thereof for the treatment of various types of skin disease, preferably atopic dermatitis in infants, children, adults and elderly patients.

### DETAILED DESCRIPTION OF THE INVENTION

The term “composition” or “ novel pharmaceutical composition” or “a low dose topical composition” as used herein synonymously include topical dosage forms such as cream, ointment, gel, lotion, emulsion, solution or the like.

The term “composition”, as in pharmaceutical composition, is intended to encompass a drug product comprising triamcinolone acetonide or salt thereof and other inert ingredient(s) (pharmaceutically acceptable excipient(s)).

The term “a low dose topical composition” as used herein comprises less than 250 mcg/gm quantity of triamcinolone acetonide or salt thereof in pharmaceutical composition, preferably less than 100 mcg/gm quantity of triamcinolone acetonide or salt thereof, more preferably less than 50 mcg/gm quantity of triamcinolone acetonide or salt thereof, further more preferably less than 25 mcg/gm quantity of triamcinolone acetonide or salt thereof.

The present invention relates to a novel pharmaceutical composition comprising triamcinolone acetonide or salt thereof and at least one pharmaceutically acceptable excipient.

The term “stable” as used herein, refers to any composition comprising a drug having sufficient physical and chemical stability at the time of manufacturing and during the shelf life of the composition.

The term “Atopic dermatitis (AD)” as used herein, refers to eczema or atopic eczema, it is a type of inflammation of the skin (dermatitis).

The term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder or delaying, inhibiting or decreasing the likelihood of the progression of the disease, disorder or syndrome.

“Pharmaceutically acceptable excipient/excipients” are components that are added to the pharmaceutical composition other than the active ingredient triamcinolone acetonide or salt thereof.

Excipients may be added to facilitate manufacture, enhance stability, enhance product characteristics, enhance patient acceptability etc. Pharmaceutically acceptable excipient includes, but not limited to, bases, emollients, thickening agent, solubilizing agent, emulsifying agent, preservative, antioxidant, humectant, absorbent, penetration/permeation enhancer, opacifying agent, chelating agent, gelling agent, acidifying or alkalizing or buffering agent, perfumes or fragrances, antifoaming agent, adherent agent, bio adhesive polymer and vehicle and any other excipient known to the art for making pharmaceutical composition.

The term “Skin diseases” is broad ranges of medical conditions that affects the skin, hair, nails and related muscle and glands and include diseases caused by genetics, immune system dysfunction, bacterial infections, viral infections, fungal infections, allergic reactions, skin cancers, and parasites.

The use of the terms “a” and “an” and “the” and similar references in the context of describing the invention (especially in the context of the following claims) are to be construed to cover both the singular and the plural, unless otherwise indicated herein or clearly contradicted by context.

In one embodiment, an invention provides a low dose topical composition comprising:
a) Triamcinolone acetonide or salt thereof; and
b) One or more suitable pharmaceutically acceptable excipient.

In one embodiment, a low dose topical composition comprising:
a) Triamcinolone acetonide or salt thereof;
b) One or more suitable pharmaceutically acceptable excipient; and
Wherein said low dose topical composition comprises triamcinolone acetonide or salt thereof in an amount of less than 250 mcg/gm.

The inventors of the present invention surprisingly found that the use of a low dose topical composition comprising triamcinolone acetonide or salt thereof in an amount of less than 250 mcg/gm is useful for treating the various skin diseases, preferably atopic dermatitis in infants, children, adults and elderly patients, Further inventors of the present invention successfully addressed and overcome the challenges and problems associated with formulating and manufacturing low-dose triamcinolone acetonide dosage forms.

The inventors of the present invention surprisingly found that a low dose topical composition comprising triamcinolone acetonide or salt thereof exhibit qualities such as but not limited to stability, easy administration, minimum side effects, high therapeutic efficiency, and better patient compliance.

In another embodiment, an invention also relates to a process for the preparation of a low dose topical composition comprising triamcinolone acetonide or salt thereof.

In another embodiment, a low dose topical composition of the present invention may comprises less than 250 mcg/gm quantity of triamcinolone acetonide or salt thereof in pharmaceutical composition, preferably less than 100 mcg/gm quantity of triamcinolone acetonide or salt thereof, more preferably less than 50 mcg/gm quantity of triamcinolone acetonide or salt thereof, further more preferably less than 25 mcg/gm quantity of triamcinolone acetonide or salt thereof.

Another embodiment of an invention provides the process for the preparation of a low dose topical composition comprising:
a) Dispensing following oily excipients isopropyl myristate, glyceryl monostearate and white soft paraffin in vessel I;
b) Dispensing the following aqueous excipients polysorbate 40 and purified water in vessel II;
c) Dispensing the following excipients methyl paraben, propylene glycol in vessel III; wherein methyl paraben is dissolved in propylene glycol to form a clear solution;
d) Dispensing the following active and excipients triamcinolone acetonide or salt thereof, propylene glycol in vessel IV; wherein triamcinolone acetonide or salt thereof is dissolved in propylene glycol to form clear solution;
e) Adding content of step (c) into content of step (b) and stirring to form uniform and homogeneous emulsion;
f) Heating content of step (b) and step (a) at about 75° C. and stirring to form a homogenous uniform emulsion;
g) Cooling the above emulsion gradually to temperature of about 25° C.-30° C.
h) Adding the content of step (d) to the primary emulsion of (f) with constant stirring; and
i) Making up the volume of the emulsion with purified water to the required quantity.

In another embodiment, present invention can be prepared in the pharmaceutical formulation such as but not limited to cream, ointment, gel, paste, lotion, film, patch, spray, foam, solution, emulsion, microemulsions, nano emulsion, emulgel, suspension, powder or the like or any combination thereof.

In another embodiment, present invention comprises one or more pharmaceutically acceptable excipient such as but not limited to bases, emollients, thickening agent, surfactants, penetration enhancer, solubilizing agent, emulsifying agent, preservative, antioxidant, humectant, absorbent, opacifying agent, chelating agent, gelling agent, acidifying or alkalizing or buffering agent, perfumes or fragrances, antifoaming agent, adherent agent, bio adhesive polymer and vehicle or the like or any combination thereof.

In another embodiment, present invention comprises one or more of bases includes but not limited to white soft paraffin, carnauba wax, cetyl alcohol, cetyl ester wax, emulsifying wax, hydrous lanolin, lanolin, lanolin alcohols, vegetable oils and animal fat; coconut oil, bees wax, olive oil, spermaceti wax, squalene, sesame oil, almond oil, alcohols, acids and esters; oleic acid, oleyl alcohol, palmitic acid, lauryl alcohol, lauric acid, myristyl alcohol, ethyl oleate, isopropyl myristate, ethylene glycol, hydrogenated and sulphated oils; hydrogenated castor, cotton seed, hydrogenated sulphated castor oils, microcrystalline wax, liquid paraffin, petrolatum, polyethylene glycol, stearic acid, stearyl alcohol, white wax, yellow wax or mixture thereof.

In another embodiment, present invention comprises one or more of emollients include but not limited to waxes, fats, caprylic/capric triglyerides, castor oil, ceteareth-20, ceteareth-30, cetearyl alcohol, ceteth 20, cetostearyl alcohol, cetyl alcohol, cetyl stearyl alcohol, cocoa butter, di-isopropyladipate, glycerin, glyceryl monooleate, glyceryl monostearate, glyceryl stearate, isopropyl myristate, isopropyl palmitate, lanolin, lanolin alcohol, hydrogenated lanolin, liquid paraffins, linoleic acid, mineral oil, oleic acid, white petrolatum, polyethylene glycol, polyoxyethylene glycol fatty alcohol ethers, polyoxypropylene 15-stearyl ether, propylene glycol stearate, squalane, steareth-2 or -100, stearic acid and urea, oils of natural origin such as almond oil, coconut oil, olive oil, palm oil, peanut oil and the like, fatty acids such as lauric acid, myristic acid, palmitic acid, and stearic acid, monohydric alcohol esters of the fatty acids such as ethyl laurate, isopropyl laurate, ethyl myristate, n-propyl myristate, ethyl palmitate, methyl palmitate, methyl stearate, ethyl stearate, isopropyl stearate, butyl stearate, isobutyl stearate, amyl stearate, and isoamyl stearate, glycols such as ethylene glycol, diethylene glycol, polyethylene glycol, branched aliphatic alcohols such as lauryl alcohol, myristyl alcohol, and stearyl alcohol or any mixture thereof.

In another embodiment, present invention comprises one or more of surfactant includes but not limited to ionic, cationic and Non-ionic surfactants or the like or any mixture thereof, further one or more of surfactant selected from but not limited to polysorbate, polysorbate 20, polysorbate 40, polysorbates 80, polysorbate 60, poloxamer, sorbitan monostearate, sorbitan monooleate, sodium lauryl sulfate, propylene glycol monostearate, dodecyl benzene sulfonate, polyethylene glycol, PEG 100 stearate polyethylene glycol 6000 distearate, polyethylene glycol 1000 monocetyl ether, polyethylene glycol monostearate, polyethylene glycol 400 monostearate, polyoxyethylene glycol fatty alcohol ethers, polyoxyl 20 cetostearyl ether, polyoxyl 40 stearate, PPG-26 oleate, sorbitan esters, sorbitan monolaurate, sorbitan monopalmitate, sorbitan palmitate, sorbitan sesquioleate, or the like or mixture thereof.

In another embodiment, present invention comprises one or more of solubilizing or emulsifying agent includes but not limited to polysorbate, polysorbate 20, polysorbate 40, polysorbates 80, polysorbate 60, poloxamer, emulsifying wax, sorbitan monostearate, sorbitan monooleate, sodium lauryl sulfate, propylene glycol monostearate, natural gums like acacia and tragacanth, monovalent and bivalent soaps, lanolin, cholesterol or cholesterol esters, triethanolamine and its salts, dodecyl benzene sulfonate, diethylene glycol monoethyl ether, docusate sodium, aluminum starch octenyl succinate, ammonium hydroxide, amphoteric-9, beeswax, synthetic beeswax, carbomer 934, carbomer 934P, carbomer 940, ceteareth-20, ceteareth-30, cetearyl alcohol, ceteth20, cetyl alcohol, cholesterol, cyclomethicone, diglycerides, dimethicone (e.g., dimethicone 350), disodium monooleamidosulfosuccinate, fatty acid pentaerythritol ester, glycerides, glyceryl monooleate, glyceryl monostearate, lanolin alcohol, hydrogenated lanolin, magnesium stearate, mineral oil, mono glycerides, polyethylene glycol, PEG 100 stearate polyethylene glycol 6000 distearate, polyethylene glycol 1000 monocetyl ether, polyethylene glycol monostearate, polyethylene glycol 400 monostearate, polyoxyethylene glycol fatty alcohol ethers, polyoxyl 20 cetostearyl ether, polyoxyl 40 stearate, PPG-26 oleate, quaternium-15, simethicone, sorbitan esters, sorbitan monolaurate, sorbitan monopalmitate, sorbitan palmitate, sorbitan sesquioleate, steareth-2, steareth-100, stearic acid, stearyl alcohol and Trolamine or mixture thereof.

In another embodiment, present invention comprises one or more of preservative includes but not limited to chorocresol, benzoic acid, phenyl mercuric nitrate, benzyl alcohol, potassium sorbate, benzoic acid and its salts, boric acid, methyl paraben, propyl paraben, trihydrate and anhydrous sodium acetate, chlorhexidine, formaldehyde, glutaraldehyde, imidazolidinyl urea, trichlosan, benzalkonium chloride, chloroxylenol, cetrimonium chloride, sodium edetate or any mixtures thereof.

In another embodiment, present invention comprises one or more of vehicle includes but not limited to purified water, hexylene glycol, oleyl alcohol, propylene carbonate, mineral oil, almond oil, cottonseed oil, ethyl oleate, isopropyl myristate, isopropyl palmitate, myristyl alcohol, Octyldodecanol, olive oil, Peanut oil, sunflower oil, sesame oil, soybean oil, squalene, polyethylene glycol, diethylene glycol monoethyl, glycofurol, benzyl alcohol, labrasol, benzyl benzoate and ethyl oleate, diethylene glycol monoethyl ether, ethyl alcohol, cremophore ELP, solutol, transcutol, capmul, captex, castor oil, di-isopropyl adipate, fatty alcohol citrate, glycerin, 1,2,6-hexanetriol, isopropyl alcohol, phosphoric acid, monocetyl ether, polyethylene glycol monostearate, polyoxyl 20 cetostearyl ether, polyoxypropylene 15-stearyl ether, polysorbates, and SD alcohol 40, triglycerides of saturated fatty acids, fatty esters of natural fatty acids, triglycerides of animal or vegetable, medium chain triglycerides, mixtures of mono-, di- and/or triglycerides, waxes, hydrogenated vegetable oils, lanolin oil, citrate tri isocetyl triglycerides having 10-18 carbon atoms, caprylic/capric triglycerides, coconut oil, corn oil, linseed oil, oil of mink, palm oil, diethylene glycol monomethyl ether, saturated paraffin oils, light or heavy vegetable oils or glycerides or mixture thereof.

In another embodiment, present invention comprises one or more of thickening agent include but not limited to carbomer, hydrogenated castor oil, methyl cellulose, sodium carboxyl methyl cellulose, carrageenan, colloidal silicon dioxide, natural gum such as gelatin, tragacanth gum, xanthan gum and guar gum, hydroxypropyl cellulose, hydroxypropyl methyl cellulose, polyethylene oxide, Alginic acid, sodium alginate, paraffin, cetostearyl alcohol, PEG 200, PEG 300, PEG 400, PEG 600, Monoethanolamine, triethanolamine, glycerol, propylene glycol, polyoxyethylene sorbitan monoleate, and Poloxamers, polyvinyl pyrrolidone, various alcohols such as Polyvinyl alcohol, ethanol or isopropyl alcohol, fumed silica, aluminum stearate, beeswax, cetyl alcohol, cetyl esters wax, dextrin, glyceryl monostearate, kaolin, paraffin, petrolatum, polyethylene stearate, starch, stearyl alcohol, wax, white wax, and bentonite or mixture thereof.

In another embodiment, present invention comprises one or more of antioxidant includes but not limited to butylated hydroxyl anisole, butylated hydroxyl toluene, propyl gallate, polyols like sorbitol, xylitol and maltitol, natural extracts like quillaia, or lactic acid or urea, lithium chloride, ascorbic acid, sodium metabisulfite, carotenoids, carotenes such as α-carotene, β-carotene and lycopene, chlorogenic acid, tocopherols, uric acid, zinc oxide, zinc sulfate, glutathione, lipoic acid, ubiquinol, sodium benzoate, and tertiary-butyl hydroquinone or mixture thereof.

In another embodiment, present invention comprises one or more of humectant includes but not limited to glycerin, glyceryl triacetate, propylene glycol, polyethylene glycol, sorbitol solution, 1,2,6 hexanetriol, isopropyl myristate, petrolatum, isopropyl palmitate, hydrogenated castor oil, mineral oil, polyoxymethylene urea, potassium sorbate or mixture thereof.

In another embodiment, present invention comprises one or more of absorbent includes but not limited to magnesium carbonate, calcium carbonate, starch, cellulose and its derivatives or mixture thereof.

In another embodiment, present invention comprises one or more of penetration enhancer includes but not limited to propylene glycol, ethanol, isopropyl alcohol, oleic acid, polyethylene glycol, phospholipids, cyclodextrins, black pepper (Piper nigrum), pyrrolidones, dimethyl sulphoxide (DMSO), decylmethyl sulphoxide (DCMS), terpenes (cineole, eugenol, d-limonene, menthol, menthone), urea, polyethylene glycol monolaurate, and butanediol; ethers, including diethylene glycol mono ethyl ether and diethylene glycol monomethyl ether; fatty acids, including lauric acid, oleic acid, and valeric acid; fatty acid esters, including isopropyl myristate, isopropyl palmitate, methyl propionate, and ethyl oleate; dimethyl acetamide, dimethylformamide 2-pyrrolidone, ethanolamine, methyl-2-pyrrolidone, diethanolamine, and triethanolamine; alkanones; organic acids, including salicylic acid, citric acid, and succinic acid; or any mixtures thereof.

In another embodiment, present invention comprises one or more of opacifying agent include but not limited to higher fatty alcohols such as cetyl, stearyl, cetostearyl alcohol, Arachidyl and behenyl alcohols, solid esters such as cetyl palmitate, glyceryl laurate, various fatty acid derivatives such as propylene glycol and polyethylene glycol esters, inorganic materials such as magnesium aluminum silicate, zinc oxide, titanium dioxide or other sunblocking agents or mixture thereof.

In another embodiment, present invention comprises one or more of chelating agent includes but not limited to ethylene diamine tetra acetate, dimercaprol, dimercaptosuccinic acid, penicillamine, deferoxamine, deferasirox, citric acid, maleic acid, phosphoric acid and like or mixture thereof.

In another embodiment, present invention comprises one or more of gelling agent includes but not limited to carbomer/carbopols, Pemulen®, cellulose derivatives such as methyl cellulose, hydroxy ethylcellulose, hydroxy propyl methylcellulose, carboxy methyl cellulose, hydroxy propyl cellulose, glycerol, or propylene glycol gelled with suitable agents such as natural gums such as xanthan gum and tragacanth, fenugreek mucilage, pectin, poloxamers (pluronics), alginate, gelatin, starch, polyvinyl alcohol, povidone, talc, clays, vegetable colloids, carboxypolymethylene or mixture thereof.

In another embodiment, present invention comprises one or more of agent includes but not limited to acidifying or alkalizing or buffering agent includes but not limited to anhydrous and monohydrate citric acid, tartrate buffer, phosphoric acid, sodium hydroxide, potassium hydroxide, ammonium hydroxide, sodium bicarbonate, potassium sorbate, monobasic and dibasic sodium phosphate, trolamine, triethanolamine or mixture thereof.

In another embodiment, present invention comprises perfumes or fragrances includes but not limited to lavender, lemon, gardenia, hypo allergenic perfume, menthol or mixture thereof.

In another embodiment, present invention comprises one or more of an antifoaming agent includes but not limited to cyclomethicone, dimethicone and simethicone or mixture thereof.

In another embodiment, present invention comprises one or more of adherent agent includes but not limited to natural gum (arabic, align, guar, tragacanth and xanthan gums, pectin and dextran) or mixture thereof.

In another embodiment, bio-adhesive polymer includes but not limited to carbomer, ethyl cellulose, polyvinylpyrrolidone, hydroxyl propyl cellulose, hydroxyl propyl methyl cellulose, hydroxyl ethyl cellulose or mixture thereof.

The concentration of active ingredient triamcinolone acetonide or salt thereof and also of other excipients, manufacturing process has been optimized in way such that composition will have maximum therapeutic efficacy in the treatment of topical skin disorder with minimum or no side effects as well as minimum or no toxicity in pediatric patients compared to the commercially available or product known in the prior art which results in better patient compliance.

In another embodiment, a low dose topical composition comprising triamcinolone acetonide or salt thereof according to present invention may be manufactured by trituration method, levigation method, fusion method, chemical reaction method or emulsification method, cold method, dispersion method, moulding, compression, hand rolling and shaping, pour moulding or the like or any combination thereof.

In another embodiment, low dose topical composition comprising triamcinolone acetonide or salt thereof according to present invention may be packaged into the collapsible tubes, jars, pot, bottle or single dose packets. The container material or packaging material of the present invention does not affect the quality of the preparation or does not allow diffusion of any kind into or across the material of the container into the preparation.

In another embodiment, a low dose topical composition comprising triamcinolone acetonide or salt thereof according to present invention may be loaded for stability studies as per ICH guidelines.

In another embodiment, a low dose topical composition comprising triamcinolone acetonide or salt thereof according to present invention may be labeled according to the requirement under Good Manufacturing Practice.

The following examples serve to illustrate the embodiments of the present invention. However, they do not intend to limit the scope of the invention.

### Examples

The following table 1 shows cream formulation containing 100.00 mcg per gm, 50.00 mcg per gm and 25.00 mcg per gm of Triamcinolone Acetonide

TABLE 1
cream

| | Drug Strength | 100 mcg/gm | 50 mcg/gm | 25 mcg/gm |
|---|---|---|---|---|
| Sr. | | % (w/w)/ | % (w/w)/ | % (w/w)/ |
| No. | Ingredients | gm | gm | gm |
| 1 | Triamcinolone Acetonide | 10.00 mg* | 5.00 mg* | 2.50 mg** |
| 2 | White Soft Paraffin | 0.5-20 | 0.5-20 | 0.5-20 |
| 3 | Glyceryl MonoStearate | 5-35 | 5-35 | 5-35 |
| 4 | Isopropyl Myristate | 2-10 | 2-10 | 2-10 |
| 5 | Propylene Glycol | 1-25 | 1-25 | 1-25 |
| 6 | PolySorbate 40 | 0.5-20 | 0.5-20 | 0.5-20 |
| 7 | Methyl Paraben | 0.05-2 | 0.05-2 | 0.05-2 |
| 8 | Purified Water | q.s. | q.s. | q.s. |

100.00 mcg per gm and for 100 gm, it is 10.00 mg*
50.00 mcg per gm and for 100 gm, it is 5.00 mg*
25.00 mcg per gm and for 100 gm, it is 2.50 mg**

### Manufacturing Process

a) Dispensing following excipients—isopropyl myristate, glyceryl monostearate and white soft paraffin in vessel I;
b) Dispensing the following excipients—polysorbate 40 and purified water in vessel II;
c) Dispensing the following excipients methyl paraben, propylene glycol in vessel III; wherein methyl paraben is dissolved in propylene glycol to form a clear solution;
d) Dispensing the following active & excipients triamcinolone acetonide or salt thereof, propylene glycol in vessel IV; wherein triamcinolone acetonide or salt thereof is dissolved in propylene glycol to form clear solution;
e) Adding content of step (c) into content of step (b) and stirring to form uniform and homogeneous emulsion;
f) Heating content of step (b) and step (a) at about 75° C. and stirring to form a homogenous uniform emulsion;
g) Cooling the above emulsion gradually to temperature of about 25° C.-30° C.
h) Adding the content of step (d) to the primary emulsion of (f) with constant stirring; and
i) Making up the volume of the emulsion with purified water to the required quantity.

1. A low dose topical composition comprising:
a) Triamcinolone acetonide or salt thereof;
b) One or more suitable pharmaceutically acceptable excipient;
Wherein said low dose topical composition comprises triamcinolone acetonide or salt thereof in an amount less than 250 mcg/gm.

2. The low dose topical composition comprising as claimed in claim 1, wherein dosage amount of triamcinolone acetonide or salt thereof ranges from about less than 25 mcg/gm to about less than 250 mcg/gm.

3. The low dose topical composition as claimed in claim 1, comprising suitable pharmaceutically acceptable excipient selected from group comprising of bases, emollients, thickening agent, surfactants, penetration enhancer, solubilizing agent, emulsifying agent, preservative, antioxidant, humectant, absorbent, opacifying agent, chelating agent, gelling agent, acidifying or alkalizing or buffering agent, perfumes or fragrances, antifoaming agent, adherent agent, bio adhesive polymer and vehicle and the like.

4. The low dose topical composition as claimed in claim 1 can be prepared in the pharmaceutical formulation such as but not limited to cream, ointment, gel, paste, lotion, film, patch, spray, foam, solution, emulsion, microemulsions, nano emulsion, emulgel, suspension, powder or the like and any combination thereof.

5. The low dose topical composition as claimed in claim 4, wherein the topical composition is a cream.

6. A low dose topical composition for treating skin diseases, comprising a) Triamcinolone acetonide or salt thereof; b) One or more suitable pharmaceutically acceptable excipient; Wherein said low dose topical composition comprises triamcinolone acetonide or salt thereof in an amount less than 250 mcg/gm.

7. The low dose topical composition as claimed in claim 1, wherein the topical composition can be useful in the treatment of infants, children, adults and elderly patients suffering from various skin diseases.

8. The low dose topical composition as claimed in claim 7, wherein the skin disease is atopic dermatitis.

9. The low dose topical composition as claimed in claim 6, wherein the topical composition can be useful in the treatment of infants, children, adults and elderly patients suffering from various skin diseases.

10. The low dose topical composition as claimed in claim 9, wherein the skin disease is atopic dermatitis.
